(19) United States
(12) Patent Application Publication
(10) Pub. No.: US 2005/0075608 A1
(43) Pub. Date: Apr. 7, 2005

(54) Medicament delivery device

(75) Inventors: James Alistair Holdgate, Warwickshire (GB); Robert Frederick Veasey, Warwickshire (GB); Christopher Nigel Langley, Warwickshire (GB)

Correspondence Address: OLIFF & BERRIDGE, PLC, P.O. BOX 19928, ALEXANDRIA, VA, 22320, US

(73) Assignee: DCA Design International Limited, Warwick (GB)

(21) Appl. No.: 10/375,098

(22) Filed: Feb. 28, 2003

(30) Foreign Application Priority Data
Feb. 28, 2002 (GB) 0204673.8

(51) Int. Cl. A61M 1/00

(52) U.S. Cl. 604/151

(57) ABSTRACT

An actuator is provided which is adapted, in conjunction with a cartridge, to form a needle-less injector, the cartridge being filled with a liquid to be injected in a subject, and having a liquid outlet and a free piston in contact with the liquid. The actuator comprises an impact member urged by a spring and temporarily restrained by a latch, the impact member being movable in a first direction under the force of the spring to first strike the free piston and then to continue to move the piston in the first direction to expel a dose of liquid through the liquid outlet, the spring providing a built-in energy store. A pressure pad surrounds the said liquid outlet and, in use, bears on the subject's skin. The actuator may be formed of two main housing components urged apart by an integral cantilever spring formed on one of them, a safety catch can be provided to prevent inadvertent actuation.

### FIELD OF THE INVENTION

The present invention relates to a medicament delivery device, and in particular, but not exclusively to a medicament delivery device such as an injector for self-administration of a medicament. Such injectors include those known as pen-type injectors.

### BACKGROUND TO THE INVENTION

In the past, it was known for those requiring regular injections of a medicament, such as those having diabetes requiring infections of insulin, to use a syringe to remove a required amount of medicament from a phial, and then use the syringe to inject the required amount of medicament. There are a number of drawbacks with this method. These include the fact that the loading of the syringe in this way is a delicate and somewhat skilled task, while those with, for example diabetes, tend to be infirm or have impaired vision, each of which increases the risk of inadvertent pricking by the syringe. In addition, the use of syringes in public places has increasingly become socially unacceptable. To overcome these difficulties pen-type injectors have been developed.

Such injectors include within a main housing a medicament cartridge. The medicament cartridge is supplied loaded with a medicament. A user then selects a dosage and uses the pen-type injector to deliver the selected amount of medicament. The pen-type injectors are, as the name suggests, typically in the shape of a pen, though sometimes other forms are adopted. In each case, however, the appearance is non-descript so as not to draw attention to the device.

Often, the medicament cartridge is replaceable to facilitate reuse of the injector. As noted above, those using such devices are often inform or have impaired vision. There is accordingly a demand for a medicament delivery device in which a medicament cartridge can be easily removed from the medicament delivery device and equally easily replaced with a replacement medicament cartridge.

### BRIEF DESCRIPTION OF THE INVENTION

According to a first aspect of the present invention, in a medicament delivery device comprising a housing, a cartridge retainer having a bore for receiving a medicament cartridge, the cartridge retainer being adapted for pivoting movement between an open position angularly displaced form the houses and a closed position adjacent the housing, a drive means, in use, operating within the cartridge retainer to cause medicament to be expelled from a medicament cartridge and a latch for releasably retaining the cartridge retainer in the closed position.

Preferably, the latch is moveable between a first advanced position and a second retracted position. More preferably, the drive means are adapted to move the latch from the first position to the second position. Even more preferably, the drive comprises a plunger having a head, the head engaging with the latch to move the latch from the first position to the second position.

Alternatively, the latch is received within the housing and comprises spring means, and a body portion, the body portion further comprising a spring engaging portion, a downwardly extending part extending at an angle away from the spring engaging portion, a generally U-shaped portion extending from an end of the downwardly extending part remote from the spring engaging portion, a laterally extending tab extending from an end of the generally U-shaped portion remote from the downwardly extending part and a forwardly extending tab extending from a web of the generally U-shaped portion. Preferably the drive means comprises a plunger having a head, the head engaging with the laterally extending tab of the latch to move the latch from the first position to the second position. More preferably the cartridge retainer comprises a recess for mating engagement with the forwardly extending tab of the latch.

Preferably the housing includes first and second elongated slots, the cartridge retainer comprises first and second tabs, each of the first and second tabs having fingers extending therefrom, in use the tabs extending through the slots and the open position is achieved when the fingers abut against a rear surface of the housing adjacent the slots.

### BRIEF DESCRIPTION OF THE DRAWINGS

The invention will now be described, by way of example only, with reference to the accompanying drawing, in which:

FIG. 1 shows a sub assembly incorporating a housing, a cartridge retainer for a medicament cartridge and a latch in accordance with the present invention, with the cartridge retainer in an open position;

FIG. 2 shows the sub-assembly of FIG. 1, with the cartridge retainer in a partially closed position;

FIG. 3 shows the sub-assembly of FIGS. 1 and 2, with the cartridge retainer in a closed position;

FIG. 4 shows the sub-assembly in the position shown in FIG. 3, but from a different angle;

FIG. 5 shows an enlarged view of the latch;

FIG. 6 shows a view of the housing without the cartridge retainer or latch;

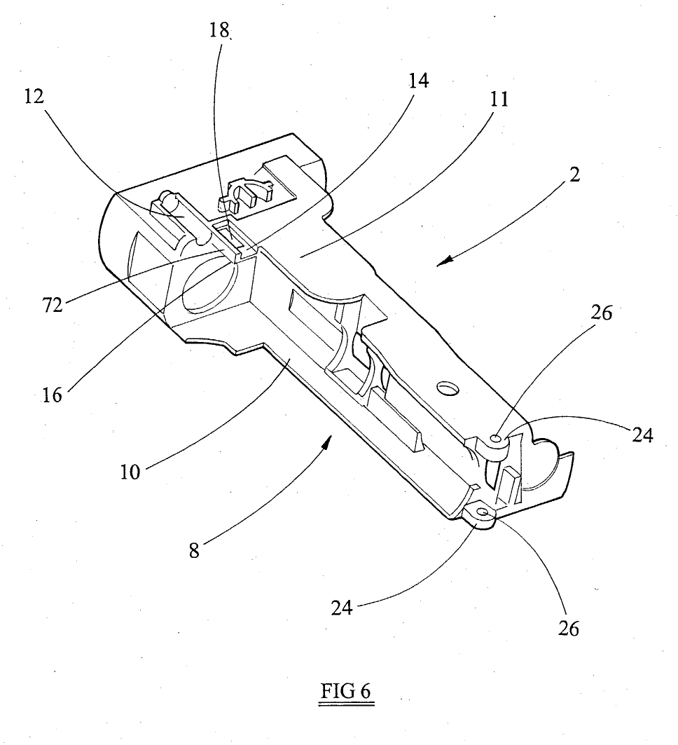

FIG. 7 shows a view of the cartridge without the housing or the latch;

FIG. 8 shows a view of a second embodiment of the latch;

FIG. 9 shows a view of a second embodiment of the housing for use with the latch shown in FIG. 8;

FIG. 10 shows a detail of a third embodiment of a sub-assembly incorporating a housing, a cartridge retainer for a medicament cartridge and a latch in accordance with the present invention; and

FIG. 11 shows the sub-assembly of FIG. 10 as a whole.

### DETAILED DESCRIPTION OF THE INVENTION

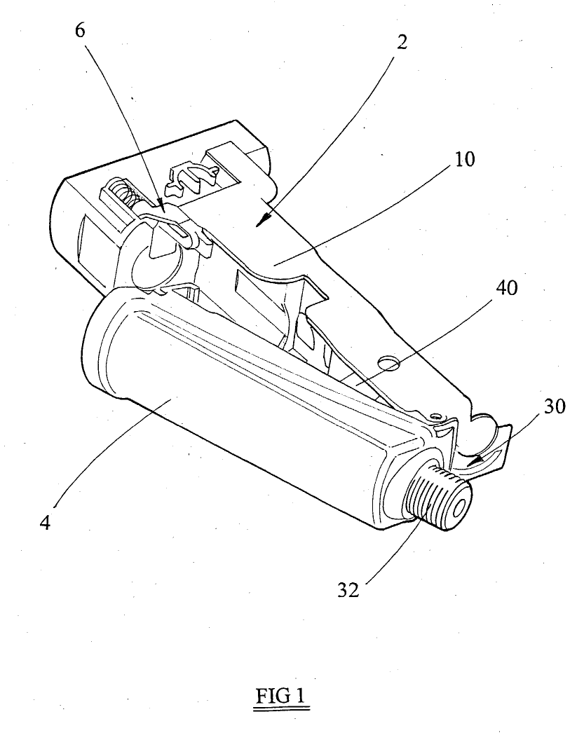

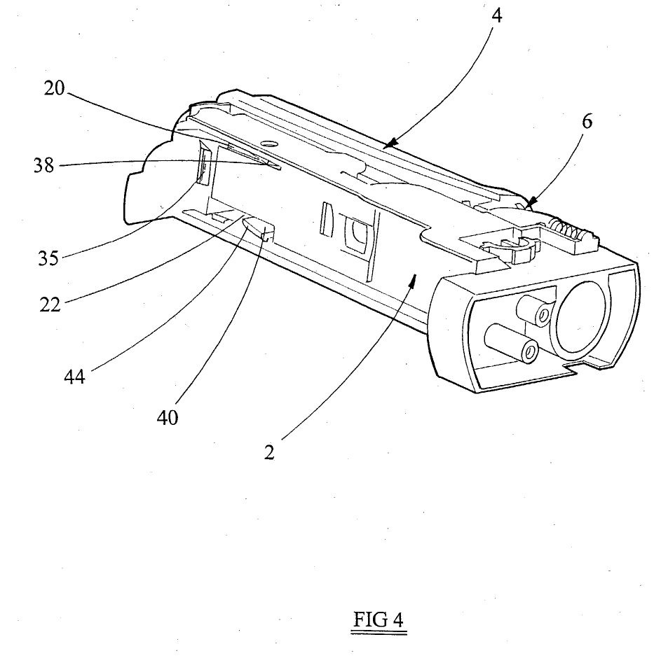

Referring first to FIGS. 1 to 4, there may be seen a sub-assembly incorporating a housing 2, a cartridge retainer 4 for a medicament cartridge and a latch 6. A medicament delivery device will also typically comprise a number of further elements such as a cover, which may or may not be integrated with the housing, a drive means and a microprocessor to store information and to orate electrical aspects of the medicament delivery device such as a display or motor means for operation of the drive means. This will be understood by one skilled in the art. However, since such elements do not form part of the present invention they are not discussed further herein.

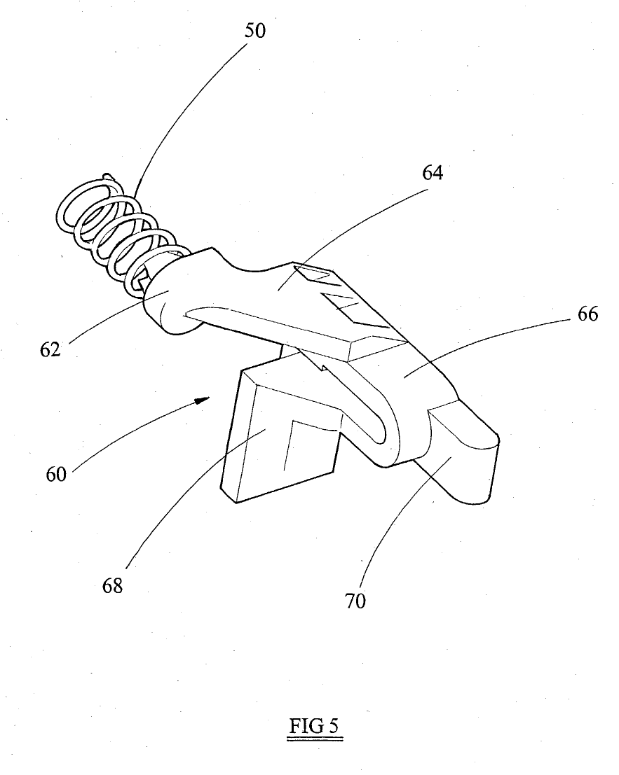

The housing 2 (shown separately in FIG. 5) comprises a generally elongate structure disposed along a first elongate axis having a first end and a second end. Intermediate the first end and the second end there is disposed a cut-away or recessed portion 8 extending from the second end towards the first end. First and second shoulders 10, 11 extend along the sides of the recessed portion 8.

At the first end of the recessed portion 8 of the housing 4 there are provided a first shaped grove 12 and a second shaped groove 14. A projecting part 16 located adjacent the first shaped groove 12 extends within the recessed portion 8 from the first end of the housing 2 towards the second end of the housing 2. The second shaped groove extends within the projecting part 16. The projecting part is further provided with an opening 18 connecting the second shaped groove 14 with the recessed portion 8. The recessed portion 8 is further provided with first and second elongate slots 20,22.

At the second end of the housing 2 there are provided two oppositely disposed projections 24. Each of the projections 24 is provided with a through bore 26.

The cartridge retainer 4 comprises a body portion of generally elongate part-cylindrical configuration and is provided with an internal cylindrical bore (not shown), adapted in use, to receive a medicament cartridge (not shown). The cartridge retainer 4 is provided at a second end with a boss 30 having a thread 32 about an external portion thereof. In use, a needle unit (not shown) will be threadably engaged with the external thread 32 of the boss 30.

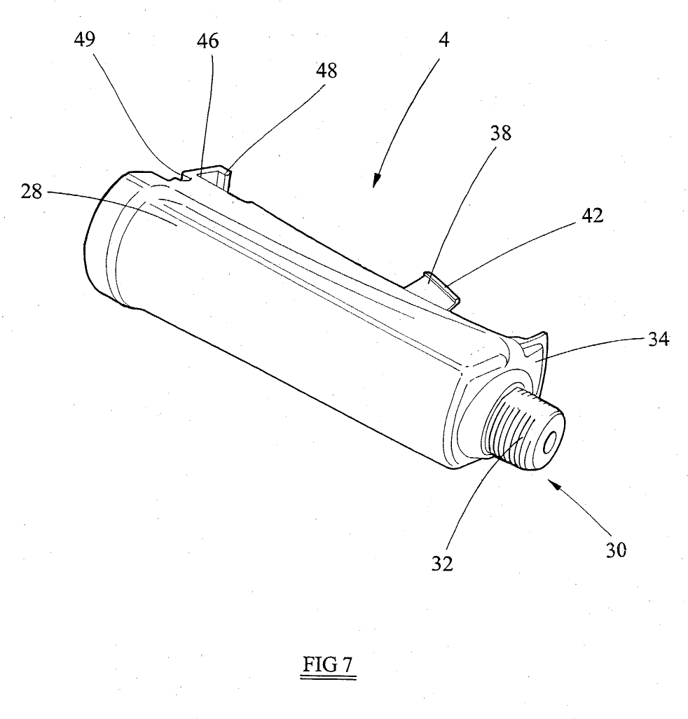

The cartridge retainer 4 (shown separately in FIG. 7) is further provided with an outwardly directed flange 34 extending from one side of a body 28 of the cartridge retainer 4. The flange 34 is located towards the second end of the cartridge retainer 4. At an end remote from the body 28 of the cartridge retainer 4 the flange 34 is provided with a barrel portion 35 having a through bore. In use the through bore of the barrel portion 35 is aligned with the through bores 26 of the projections 24 formed at the second end of the housing 2 and pivot pin 36 passed through the aligned bores. In this way, the cartridge retainer 4 is adapted to pivot with respect to the housing 2.

The cartridge retainer 4 is further provided with first and second oppositely disposed tabs 38, 40, each of the first and second tabs 38, 40 extending away from the body 28 of the cartridge retainer 4. The first and second tabs 38, 40 are located approximately one third along the cartridge retainer 4 towards the second end of the cartridge retainer 4. Each of the first and second tabs 38,40 is provided with an outwardly directed finger 42, 44 that is each finger is disposed in a direction away from the other of the first and second tabs 38, 40.

In use, each of the first and second tabs 38,40 is adapted to extend through a respective one of the first and second elongate slots 20,22 provided in the housing 2. The outwardly directed fingers 42,44 will locate behind respective edges of the first and second elongate slots 20,22 to prevent pivotal movement of the cartridge retainer 2 with respect to the housing 2 beyond a predetermined position.

At the first end of the cartridge retainer 2 there is provided a further outwardly directed flange 46. The further flange 46 is provided with a ridge or projection 48 extending away from the further flange 46 in a direction generally parallel to the elongate axis of the cartridge retainer 4. At the second end of the cartridge retainer 4, between the bore of the cartridge retainer 4 and the further flange 46, a recess 49 is formed within the body 28 of the cartridge retainer 4.

Within the first and second shaped grooves 12,14 of the housing 2 there is located a latch 6 (shown separately in FIG. 5). The latch 6 comprises a means and a body portion 60. The spring means illustrated in the Figures is a helical spring 50. The spring means is located in a closed end of the first shaped groove 12. It will be understood that in the presence of a cover (not shown) the first shaped groove will become a blind bore, thereby constraining operation of the spring 50.

The body portion 60 of the latch 6 is located within both the first and second shaped grooves 12,14.

The body portion 60 of the latch 6 comprises a spring engaging portion 62, a downwardly extending part 64 extending at an angle away from the spring engaging portion 62, a generally U-shaped portion 66 extending from an end of the downwardly extending part 64 remote from the spring engaging portion 62, a laterally extending tab 68 extending from an end of the generally U-shaped portion 66 remote from the downwardly extending part 64 and a forwardly extending tab 70 extending from a web of the generally U-shaped portion 66.

In use, the spring engaging portion 62 is located within the first shaped groove 12 with the spring means. The generally U-shaped portion 66 is located about the projecting part 16 of the housing 2, the laterally extending tab 68 being disposed generally parallel to a surface of the first end of the housing abutting a side surface 72 of the projecting part 16. The body portion 60 is further provided with a protrusion (not shown) which is seated in and adapted to travel within the opening 18 in the projecting part 16.

Operation of the cartridge retainer 4 and the associated latch 6 will now be explained with principal reference to FIGS. 1 to 3. In FIG. 1, the cartridge retainer 4 is at an extreme angular location with respect to the housing 2. The cartridge retainer 4 is prevented from moving beyond this position by the restraining effect of the fingers 42,44 of the first and second tabs 38,40 abutting with the surfaces behind the respective first and second elongate slots 20,22 through which the first and second tabs 38,40 pass.

In this position, the opening of the bore of the cartridge retainer 4 at the first end of the cartridge retainer 4 is clear of the housing 4 allowing a medicament cartridge easily to be inserted into or withdrawn from the cartridge retainer 4. Preferably, the medicament cartridge should easily slide within the bore of the cartridge retainer 4 such that when the cartridge retainer 4 is in the position shown in FIG. 1, removal of a medicament cartridge may be effected simply by inverting the sub-assembly to allow the medicament cartridge to slide out due to gravity.

It will be seen that in FIG. 1 that the latch 6 is in an advanced position.

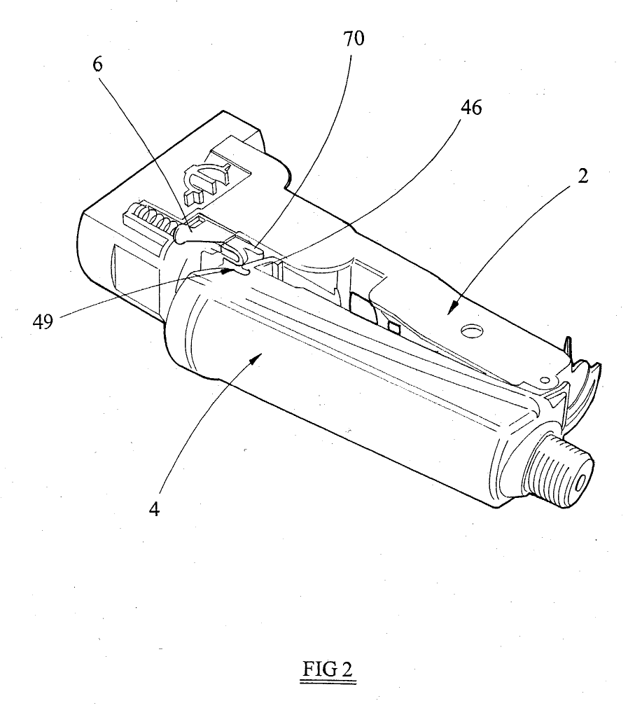

In FIG. 2, the cartridge retainer 4 has been moved to an intermediate position in which the cartridge retainer 4 is at a reduced angular displacement with respect to the housing 2. In particular, it will be noticed that the further flange 46 at the first end of the cartridge retainer 4 has pushed against the forwardly extending tab 70 of the latch 6 causing the latch 6 to be moved backwards against the spring 50 within the first shaped groove 12 in the housing 2. As the latch 6 is moved backwards, the side surface 72 of the projecting part 16 acts as a guide wall for the laterally extending tab 68.

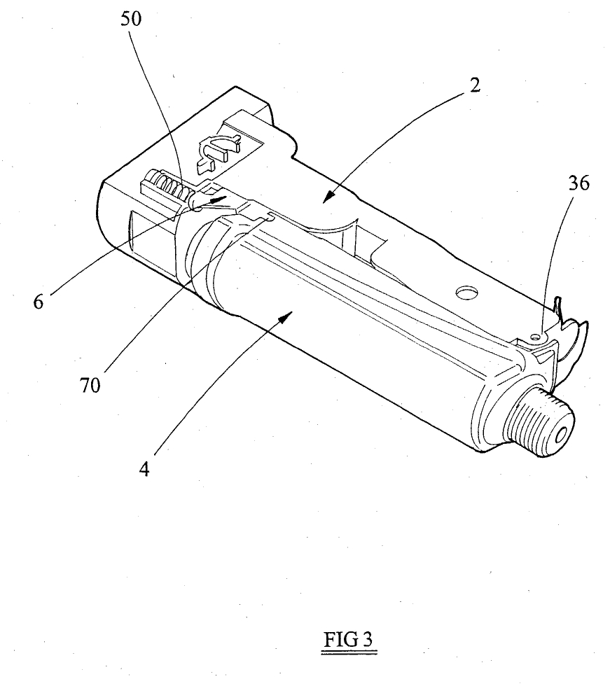

As the cartridge retainer moves from the position shown in FIG. 1 through the position shown in FIG. 2 and into the position shown in FIG. 3, the further flange 46 is received within that portion of the recessed portion 8 defined between the first and second shoulders 10,11. During this movement, it will be seen that the further flange 46 acts as a camming surface displacing the latch 6 within the first and second shaped grooves 12,14.

When the cartridge retainer 4 is the position shown in FIG. 3, a closed position, the forwardly extending tab 70 moves from the further flange 46 into the recess 49. It will be understood that the impact of the latch 6 (either the web of the U-shaped portion 6 or the forwardly extending tab 70) striking against the cartridge retainer 4 will provide an audible click, providing conformation to a user of the medicament deliver device that the cartridge retainer 4 is in a closed position against the housing 2. The forwardly extending tab 70 is held within the recess 49 under the action of the spring 50.

In one embodiment (not shown), the latch 6 is provided with a tab that extends through a slot in the cover of the medicament delivery device. A user may then manipulate the tab to withdraw the forwardly extending tab 70 of the latch from the recess 49 of the cartridge retainer 4, thereby to free the cartridge retainer 4 for pivoting movement away from the housing 2.

However, this arrangement has as a disadvantage that a user may free the cartridge retainer 4 in this way, while a drive means of the medicament delivery device is still operational within the bore of the cartridge retainer 4 to cause medicament to be expelled form the medicament cartridge. Attempting to move the cartridge retainer 4 at this time cause damage to the drive means rendering the medicament delivery device inoperative.

In a preferred embodiment, the drive means comprises a headed plunger in which the head of the plunger is located ahead of the laterally extending tab 70. When the cartridge retainer 4 is in the closed position of FIG. 3, the headed plunge is free to advance within the cartridge retainer 4. When a medicament cartridge is present within the cartridge retainer 4, this will cause medicament to be expelled from the medicament cartridge.

When a user wishes to move the cartridge retainer 4 away from the housing 2, the plunger is caused to be withdrawn from the cartridge retainer 4. This has the effect of bringing the head of the plunger into contact with the laterally extending tab 70 of the latch 6. Further movement of the plunger in this direction causes the laterally extending tab 68, together with the latch 6, to be withdrawn together with the plunger. Beyond a certain point, the forwardly extending tab 70 of the latch will be disengaged from the groove 49 in the cartridge retainer 4. This point is chosen such that at this point the head of the plunger and the laterally extending tab 68 have both been withdrawn behind the path of movement of the cartridge retainer 4. In this way, the cartridge retainer 4 cannot be moved away from the housing 2 until the drive means is clear of the cartridge retainer 4.

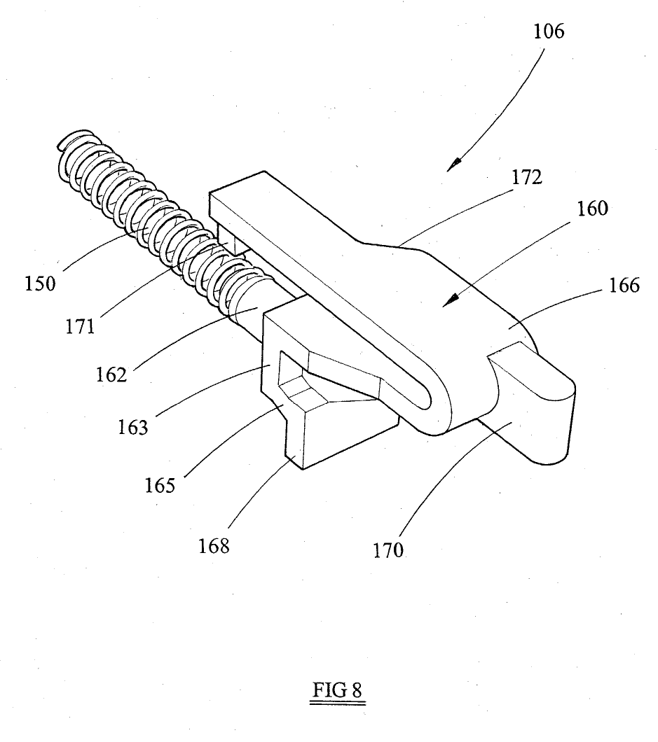

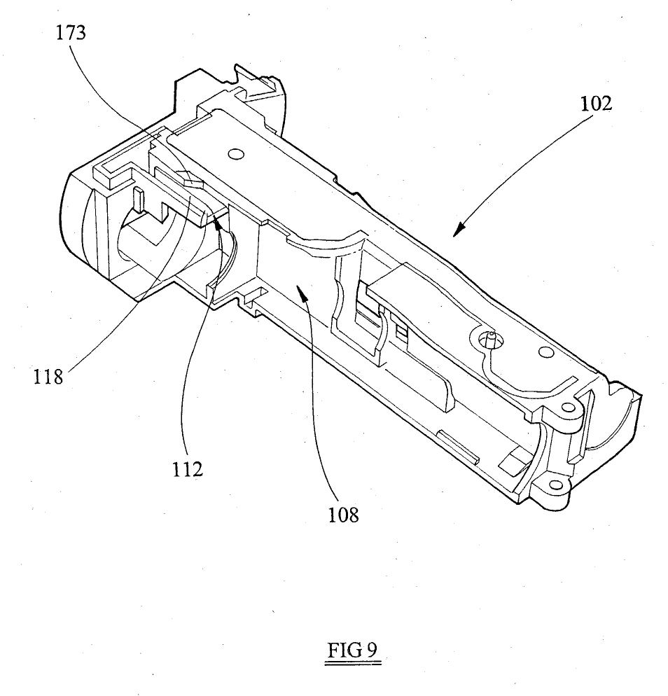

Turning now to FIGS. 8 and 9, there is shown a further embodiment of a latch and housing for use in a sub-assembly of a medicament delivery device in accordance with the present invention.

The housing 102 of this embodiment is generally similar to the housing 2 of the previous embodiment. The principal differences lie at the first end of the housing 102. The first end of a recessed portion 108 is provided with a single groove 112. The single groove 112 is formed in a region of the housing 102 of reduced thickness. The housing 102 is provided with an opening 118 extending through the single groove 112. A bore (not shown) is located in the housing 106 beneath the single groove 112.

The latch 106 comprises a spring means and a body portion 160. The spring means is conveniently in the form of a helical spring 150. In use, the spring means is located within the bore.

The body portion 160 of the latch 106 comprises a spring engaging portion 162, a web portion 163 connected across one end of the spring engaging portion 162, a forwardly directed flange 165 connected to one end of the web portion 163, a laterally extending tab 168 extending from an end of the forwardly directed flange 165 remote from the web portion 163, a generally U-shaped portion 166 extending from a second end of the web portion 163, and a forwardly extending tab 170 extending from a web of the generally U-shaped portion 166. A locating tab 171 is provided on one limb of the generally U-shaped portion 166. It can be seen that this limb is longer than the other limb of the generally U-shaped portion 166.

In use, the latch 106 is seated within the single groove 112. The locating tab 171 is adapted to be seated within the opening 118. The longer limb of the generally U-shaped portion 166 of the latch 106 is provided with a shoulder 172 which way in use abut a corresponding shoulder 173 provided on the housing 112 as part of the single groove 112.

The operation of this embodiment is essentially the same as for the previous embodiment in that as the cartridge retainer is moved to the closed position, the cartridge retainer abuts with the forwardly extending tab 170 to urge the latch 106 backwards within the single grove 112. Similarly, the drive means may comprise a headed plunger in which the head of the plunger is located ahead of the laterally extending tab 168 in order the latch 106 may be drawn backwards under the action of the plunger to allow the cartridge retainer to be pivoted away from the housing 106 to facilitate removal and/or insertion of a medicament cartridge.

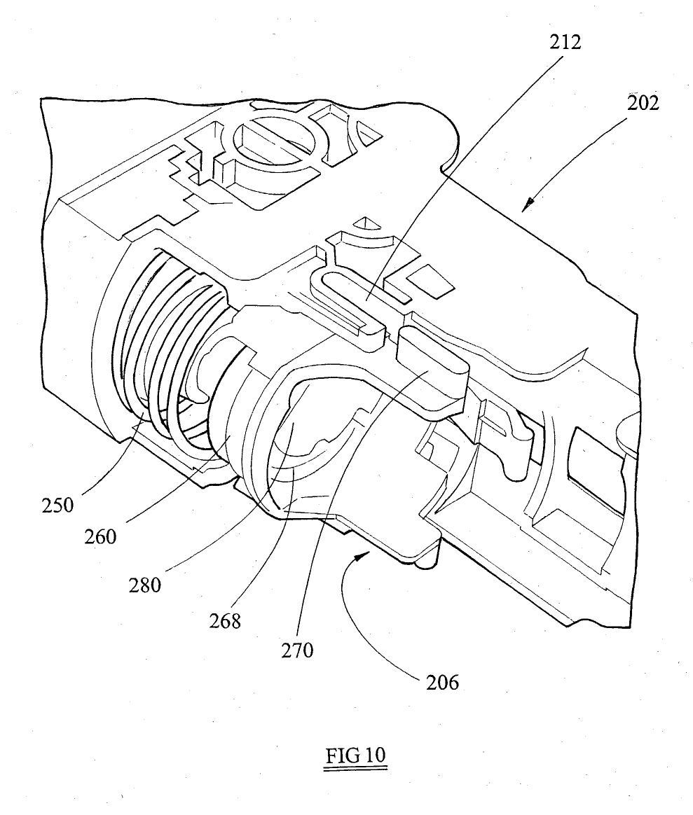

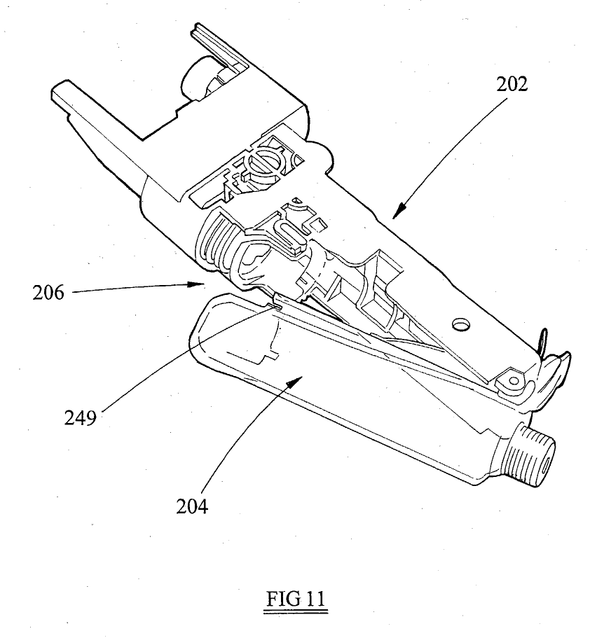

Turning now to FIGS. 10 and 11, there is shown a third embodiment of a latch 206 and housing 202 for use in a sub-assembly of a medicament delivery device in accordance with the present invention.

The housing 202 of this third embodiment is generally similar to the housing 2 of the first embodiment. The principal differences lie at the first end of the housing 202.

A slot 212 is located at a first end of the housing 202. A region of the housing 202 adjacent the slot 212 takes the form of a generally cylindrical bore.

The latch 206 comprises a spring means and a body portion 260. The spring means is conveniently in the form of a helical spring 250. In use, the spring means located within the bore. The helical spring 250 abuts against one end of the bore. This end of the bore includes an opening through which a drive means may in use extend.

The body portion 260 is generally bowl-shaped having a central opening at a base 268 of the bowl. A forwardly extending tab 270 is located at a point on a rim of the bowl. In the illustrated embodiment, the slot 212 and the forwardly extending tab 270 are each repeated about an axis of the sub-assembly. In use, a headed plunger extends through the central opening such that a head 280 of the plunger is located ahead of the base 268 of the bowl.

When the latch 206 is drawn backwards under the action of the plunger, the forwardly extending tab(s) 270 are fully retained within the slot(s) 212, and a cartridge retainer 204 is free to be pivoted away from the housing 206 to facilitate removal and/or insertion of a medicament cartridge. When the cartridge retainer 204 is in position adjacent the housing 202 the latch 206 may be released and the forwardly extending tab(s) 270 will move forward under the action of the spring 250 to engage with recess(es) 249 provided in the cartridge retainer 204 for this purpose and thereby retain the cartridge retainer 204 in position.

1. A medicament delivery device comprising a housing, a cartridge retainer having a bore for receiving a medicament cartridge, the cartridge retainer being adapted for pivoting movement between an open position angularly displaced form the housing and a closed position adjacent the housing, a drive means, in use, operating within the cartridge retainer to cause medicament to be expelled form a medicament cartridge and a latch for releasably retaining the cartridge retainer in the closed position.

2. A device according to claim 1, in which the latch is moveable between a first advanced position and a second retracted position.

3. A device according to claim 2, in which the drive means are adapted to move the latch from the first position to the second position.

4. A device according to claim 2, in which the drive means are adapted to move the latch from the first position to the second position and the drive means comprises a plunger having a head, the head engaging with the latch to move the latch from the first position to the second position.

5. A device according to claim 2, in which the latch is received within the housing and comprises spring means, and a body portion, the body portion further comprising a spring engaging portion, a downwardly extending part extending at an angle away from the spring engaging portion, a generally U-shaped portion extending from an end of the downwardly extending part remote from the spring engaging portion, a laterally extending tab extending from an end of the generally U-shaped portion remote from the downwardly extending part and a forwardly extending to extending from a web of the generally U-shaped portion.

6. A device according to claim 3, in which the latch is received within the housing and comprises spring means, and a body portion, the body portion further comprising a spring engaging portion, a downwardly extending part extending at an angle away from the spring engaging portion, a generally U-shaped portion extending from an end of the downwardly extending part remote from the spring engaging portion, a laterally extending tab extending from an end of the generally U-shaped portion remote from the downwardly extending part and a forwardly extending tab extending from a web of the generally U-shaped portion.

7. A device according to claim 5, in which the drive means comprises a plunger having a head, the head engaging with the laterally extending tab of the latch to move the latch from the first position to the second position.

8. A device according to claim 6, in which the drive means comprises a plunger having a head, the head engaging with the laterally extending tab of the latch to move the latch from the first position to the second position.

9. A device according to claim 5, in which the cartridge retainer comprises a recess for mating engagement with the forwardly extending tab of the latch.

10. A device according to claim 6, in which the cartridge retainer comprises a recess for mating engagement with the forwardly extending tab of the latch.

11. A device according to claim 7, in which the cartridge retainer comprises a recess for mating engagement with the forwardly extending tab of the latch.

12. A device according to claim 8, in which the cartridge comprises a recess for mating engagement with the forwardly extending tab of the latch.

13. A device according to claim 1, in which the housing includes first and second elongated slots, the cartridge retainer comprises first and second tabs, each of the first and second tabs having fingers extending therefrom, in use the tabs extending through the slots and the open position is achieved when the fingers abut against a rear surface of the housing adjacent the slots.
